(19) United States
(12) Patent Application Publication
(10) Pub. No.: US 2006/0069072 A1
(43) Pub. Date: Mar. 30, 2006

(54) Method of male sexual enhancement

(76) Inventor: John R. Woodward, Dallas, TX (US)

Correspondence Address: MICHAEL A. O'NEIL, P.C., 5949 SHERRY LANE, SUITE 820, DALLAS, TX, 75225, US

(21) Appl. No.: 10/952,147

(22) Filed: Sep. 28, 2004

(51) Int. Cl. A61K 31/56; A61K 31/519

(52) U.S. Cl. 514/171; 514/252.16; 514/262.1

(57) ABSTRACT

A method of sexual enhancement in men includes the steps of testing the blood of the man to determine the levels of free estradiol and free testosterone therein, assuring that the man's blood includes free estradiol within a first predetermined range and free testosterone within a second predetermined range, and thereafter administrating a drug selected from the group consisting of VIAGRA® (sildenafil citrate). LEVITRA® (vardenafil hydrochloride), and CIALIS® (tadalfil) prior to sexual activity.

### TECHNICAL FIELD

This invention relates generally to a method of sexual enhancement in men, and more particularly to a method of increasing male testosterone and estradiol levels, including the step of administering testosterone and estradiol transdermally, to facilitate the effectiveness of available drugs such as by Viagra®, Levitra®, and Cialis®.

### BACKGROUND AND SUMMARY OF THE INVENTION

As is universally known, various drugs are now available for the treatment of erectile dysfunction. By stimulating the erectile process, these drugs can promote sexual satisfaction in men. The first drug of this type to gain widespread acceptance was Viagra® which was introduced in 1998. Levitra® and Cialis® have been introduced more recently.

Viagra®, Levitra®, and Cialis® are effective in promoting the erectile process, but they are not effective when free testosterone and free estradiol levels are low. Further, these drugs do not treat the problem of male testosterone deficiency, which is a primary cause of erectile dysfunction. Other symptoms caused by male testosterone deficiency and low estradiol levels include decreased libido, fatigue, and depression. Heretofore Viagra®, Levitra®, and Cialis® have been prescribed without first bringing the free testosterone and free estradiol levels to within normal range, thus reducing the effectiveness of such drugs for increasing sexual enhancement in men.

The present invention comprises a method of treating free testosterone and free estradiol deficiency in men which overcomes the foregoing and other problems which have long since characterized male menopause. In accordance with the broader aspects of the invention, testosterone deficiency is treated by administering an H-based testosterone cream or subcutaneous testosterone pellets; estradiol deficiency is treated by administering an H-based estradiol cream or by administering commercially available Estrasorb® cream. Bringing the testosterone and estradiol levels within normal ranges, results in resolution of sexual and energy problems, helps protect against cardiovascular disease and osteoporosis, and facilitates the effectiveness of the prescription drugs used to treat erectile dysfunction.

In accordance with more specific aspects of the invention, free testosterone levels are raised to the normal range by rubbing an H-based testosterone cream into the skin of the forearms or thighs each evening or implanting a subcutaneous testosterone pellet every three months. An estradiol cream, either a topical H-based estradiol cream or commercially available Estrasorb® cream, is administered twice daily in order to raise free estradiol levels to within the normal range. The result is more effective application of the prescription drug chosen to treat erectile dysfunction and a higher level of sexual enhancement experienced by the patient.

### DETAILED DESCRIPTION

Normal free estradiol levels in men are between about 0.20 picograms per milliliter and about 0.50 picograms per milliliter. Normal free testosterone levels in men are between about 5.0 picograms per milliliter and about 20.0 picograms per milliliter.

All men should be screened to determine their free testosterone and free estradiol levels at age 50 and periodically thereafter. Free testosterone screening should be done even earlier if symptoms such as erectile dysfunction, decreased libido, fatigue, or depression are recognized. Testing of free testosterone and estradiol levels is available at Interscience Institute, Inglewood, Calif., and other laboratories.

If the free testosterone and free estradiol levels of a particular male patient is determined to be low, the method of the present invention is utilized to raise the free testosterone and free estradiol levels of the patient to the normal range. Once the testosterone and estradiol levels are elevated to within normal ranges, drugs prescribed to promote the erectile process work more effectively.

In accordance with the invention, to raise testosterone levels, testosterone is mixed with H-based cream to facilitate the transdermal administration thereof. More specifically, 50 milligrams of testosterone is mixed with 1 cc of H-based cream.

H-based cream comprises the following ingredients:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The foregoing testosterone/H-base cream mixture is administered by applying 1 cc thereof to the skin of the forearms or inner thighs each evening. Alternatively, subcutaneous testosterone pellets may be implanted every three months.

In accordance with the invention, to raise estradiol levels, estradiol is mixed with H-based cream to facilitate the transdermal administration thereof. More specifically, 3 milligrams of estradiol is mixed with 1 cc of H-based cream. Alternative to using the foregoing cream, commercially available Estrasorb® cream, manufactured by Novavax Pharmaceutical, may be used. H-based estradiol cream is administered by applying 1 cc thereof to the skin of the forearms or inner thighs once daily. Two packets of Estrasorb® cream is administered to the forearms or inner thighs once daily.

The progress of the treatment protocol is monitored by periodic retesting of the patient's free testosterone and free estradiol levels. The results achieved by means of the present method include: increased effectiveness of the prescription drugs Viagra®, Levitra®, and Cialis®, reduction or elimination of erectile dysfunction problems, increased libido, reduced fatigue, and reduction or elimination of depression.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of providing sexual enhancement in men comprising the steps of:
testing the blood of the male patient to determine the amounts of free estradiol and free testosterone therein;
assuring that the male patient's blood includes free estradiol within a first predetermined range;
assuring that the male patient's blood includes free testosterone with a second predetermined range;
thereafter administering to the male patient a predetermined amount of a drug selected from the group consisting of VIAGRA®D (sildenafil citrate). LEVITRA® (vardenafil hydrochloride, and CIALIS®D (tadalfil).

2. The method according to claim 1 wherein the step of assuring that the male patient's blood includes free estradiol within the first predetermined range includes the step of treating the male to increase the level of free estradiol in his blood.

3. The method according to claim 2 wherein the step of assuring that the male patient's blood includes free estradiol within the first predetermined range includes the step of treating the male patient with H-based estradiol cream to increase the level of free estradiol.

4. The method according to claim 2 wherein the step of assuring that the male patient's blood includes free estradiol within the first predetermined range includes the step of treating the male patient with ESTASORB® (estradiol topical emulsion) cream to increase the level of free estradiol.

5. The method according to claim 1 wherein the step of assuring that the male patient's blood includes free testosterone within the second predetermined range includes the step of treating the male patient to increase the level of free testosterone therein with H-based testosterone cream.

6. The method according to claim 5 wherein the step of assuring that the male patient's blood includes free testosterone within the second predetermined range includes the step of treating the male patient with H-based testosterone cream to increase the level of free testosterone.

7. The method according to claim S wherein the step of assuring that the male patient's blood includes free testosterone within the second predetermined range includes the step of treating the male patient with subcutaneous testosterone pellets to increase the level of free testosterone.
